(19) United States
(12) Patent Application Publication
(10) Pub. No.: US 2025/0211941 A1
(43) Pub. Date: Jun. 26, 2025

(54) PREDICTIVE GUIDANCE BASED ON SOUND MEASUREMENTS FOR ELECTRONIC DEVICES

(71) Applicant: Apple Inc., Cupertino, CA (US)

(72) Inventors: David M. FISCHER, San Francisco, CA (US); Lucas O. WINSTROM, Portola Valley, CA (US); Michael GILES, San Jose, CA (US); Michael TANNE, Alamo, CA (US); Sriram HARIHARAN, San Jose, CA (US); Suzanne C. HARDY, Los Gatos, CA (US)

(21) Appl. No.: 18/917,956

(22) Filed: Oct. 16, 2024

Related U.S. Application Data
(60) Provisional application No. 63/613,058, filed on Dec. 20, 2023.

(51) Int. Cl. H04W 4/024; G08B 21/24; H04W 4/021

(52) U.S. Cl. CPC H04W 4/024; G08B 21/24; H04W 4/021

(57) ABSTRACT

The subject technology disclosed herein may allow a device to provide guidance based on sound measurements, which may include crowd-sourced measurements of the sound level at various locations other than a current location of the user. The crowd-sourced measurements may be real time measurements and/or may be stored and/or indexed by time (day, time of day, week, month, etc.), type of activity at specific locations (happy hour, open-mic night, sports play-offs, etc.), or environmental context similar to other locations (touring band, protest, sporting event, etc.). The guidance based on the sound measurements may include a reminder notification to bring hearing protection to a location for which crowd-sourced sound level measurements have been obtained, before the user arrives at the location. The guidance may also, or alternatively, include a recommended location and/or a recommended route with a desired sound level, such as a quiet restaurant, or a quiet walking route.

### CROSS REFERENCE TO RELATED APPLICATIONS

This application claims the benefit of priority to U.S. Provisional Patent Application No. 63/613,058, entitled, “Predictive Guidance Based On Sound Measurements For Electronic Devices”, filed on Dec. 20, 2023, the disclosure of which is hereby incorporated herein in its entirety.

### TECHNICAL FIELD

The present description relates generally to electronic devices including, for example, to predictive guidance based on sound measurements for electronic devices.

### BACKGROUND

Electronic devices, including audio devices such as headphones and earbuds, can include speakers for outputting sound to a user's ears, and microphones for capturing the sound from an environment of the electronic device.

### BRIEF DESCRIPTION OF THE DRAWINGS

Certain features of the subject technology are set forth in the appended claims. However, for purpose of explanation, several embodiments of the subject technology are set forth in the following figures.

FIG. 1 illustrates an example system architecture including various electronic devices that may implement the subject system in accordance with one or more implementations.

FIG. 2 illustrates an electronic device generating predictive guidance for a location based on concurrent sound measurements by other devices at the location in accordance with one or more implementations.

FIG. 3 illustrates an electronic device generating predictive guidance for a location while on the way to the location in accordance with one or more implementations.

FIG. 4 illustrates an electronic device generating predictive guidance for a location based on prior sound measurements by other devices at the location in accordance with one or more implementations.

FIG. 5 illustrates an electronic device at a first location generating predictive guidance for a second location based on prior sound measurements at a third location in accordance with one or more implementations.

FIG. 6 illustrates an electronic device generating predictive guidance for a location based on prior sound measurements by the electronic device at the location in accordance with one or more implementations.

FIG. 7 illustrates predictive guidance implemented as a recommended route in accordance with one or more implementations.

FIG. 8 illustrates predictive guidance implemented as a recommended venue in accordance with one or more implementations.

FIG. 9 illustrates an example of a notification to bring hearing protection to a location in accordance with one or more implementations.

FIG. 10 illustrates a flow diagram for an example process for predictive guidance based on sound measurements in accordance with implementations of the subject technology.

FIG. 11 illustrates a flow diagram for another example process for predictive guidance based on sound measurements in accordance with implementations of the subject technology.

FIG. 12 illustrates an electronic system with which one or more implementations of the subject technology may be implemented.

### DETAILED DESCRIPTION

The detailed description set forth below is intended as a description of various configurations of the subject technology and is not intended to represent the only configurations in which the subject technology can be practiced. The appended drawings are incorporated herein and constitute a part of the detailed description. The detailed description includes specific details for the purpose of providing a thorough understanding of the subject technology. However, the subject technology is not limited to the specific details set forth herein and can be practiced using one or more other implementations. In one or more implementations, structures and components are shown in block diagram form in order to avoid obscuring the concepts of the subject technology.

Aspects of the disclosure may provide predictive guidance to a user of an electronic device regarding sound levels in the physical environment. The predictive guidance may include a notification to bring or wear hearing protection to a particular location. For example, the electronic device may determine that the user is predicted be at the location and that the sound level at the location is above a threshold, and responsively provide the notification to bring or wear hearing protection to the location. In some implementations, the notification is provided before the user leaves their current location (e.g., their house) for the predicted location, such that the user is able to the bring hearing protection with them.

The predictive guidance may also, or alternatively, include guidance, such as based a sound-level map, to a particular location and/or along a particular route that is based on the sound level at that particular location and/or along that particular route. The sound level at a particular location and/or along a particular route may be determined based on sound level measurements by the electronic device and/or other electronic devices. For example, the other electronic devices may be at the particular location and/or along the particular route at the time that the electronic device determines that the user is going to be the location, and/or may have been at the particular location and/or along the particular route at a similar time (e.g., the same time of day and/or the same day of the week) as the time that the electronic device determines that the user is going to be at the location. The electronic device may determine that the electronic device is going to be at the location, or along the particular route, based on user information (e.g., calendar information, motion information, purchase information, wallet information, etc.) stored at the electronic device.

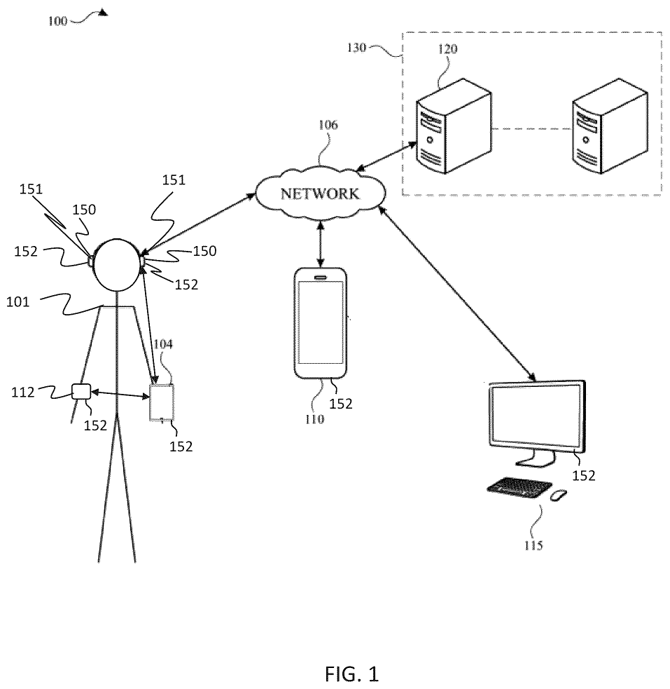

FIG. 1 illustrates an example system architecture 100 including various electronic devices that may implement the subject system in accordance with one or more implementations. Not all of the depicted components may be used in all implementations, however, and one or more implementations may include additional or different components than those shown in the figure. Variations in the arrangement and type of the components may be made without departing from the spirit or scope of the claims as set forth herein. Additional components, different components, or fewer components may be provided.

The system architecture 100 includes an audio device 150, an electronic device 104 (e.g., a handheld electronic device such as a smartphone, a tablet, or a smart watch), an electronic device 110, an electronic device 112 (e.g., a wearable electronic device, such as a smart watch or a head mountable device), an electronic device 115, and one or more servers 120 communicatively coupled by a network 106 (e.g., a local or wide area network). For explanatory purposes, the system architecture 100 is illustrated in FIG. 1 as including the audio device 150, the electronic device 104, the electronic device 110, the electronic device 112, the electronic device 115, and the server 120; however, the system architecture 100 may include any number of electronic and/or audio devices and any number of servers or a data center including multiple servers.

The audio device 150 may be implemented as an audio device such as headphones (e.g., a pair of speakers mounted in speaker housings that are coupled together by a headband), or an earbud (e.g., an earbud of a pair of earbuds, each having a speaker disposed in a housing that conforms to a portion of the user's ear) configured to be worn by a user (also referred to as a wearer when the audio device is worn by the user), or may be implemented as any other device capable of obtaining audio with one or more microphones and/or outputting audio, video and/or other types of media (e.g., and configured to be worn by a user). Each audio device 150 may include one or more speakers, such as speaker 151, configured to project sound into an car of the user 101, and one or more microphones, such as microphone 152, configured to receive audio input such as external noise input and/or external voice inputs. In one or more implementations, the audio device 150 may include multiple microphones 152 that can be co-operated to form a beamforming microphone array for obtaining sound preferentially from a particular direction and/or location.

As shown in FIG. 1, the electronic device 104, the electronic device 110, the electronic device 112, and the electronic device 115 may also each include one or more microphones 152 for obtaining audio from a physical environment around that device. The electronic device 104, the electronic device 110, the electronic device 112, the electronic device 115 may also include other components not visible in FIG. 1, including, but not limited to, processing circuitry, memory, a display for displaying electronic information visually, haptic components for generating haptic outputs (e.g., vibrations) from one or more portions of the device, communications circuitry (e.g., including wireless communications circuitry), input components, and/or output components.

Although not visible in FIG. 1, each audio device 150 may include processing circuitry (e.g., including memory and/or one or more processors, such as a central processing unit and/or one or more digital signal processors (DSPs)) and communications circuitry (e.g., one or more antennas, etc.) for receiving and/or processing audio content, such as from one or more of the electronic device 104 or the electronic device 112. The processing circuitry of the audio device 150 or another device may operate one or more speakers, such as the speaker 151, to generate sound and/or to generate noise cancellation output configured to cancel, at the user's car, one or more environmental sounds in the physical environment. In one or more implementations, the audio device 150 may be used as a hearing protection device that physically blocks, and/or acoustically cancels one or more sounds from the physical environment that are loud enough to cause hearing damage for the user 101. The memory of the audio device 150 may store one or more machine learning models implemented as neural networks and trained for one or more audio processing operations for the audio device 150.

The audio device 150, the electronic device 104, the electronic device 110, the electronic device 112, and/or the electronic device 115 may each include communications circuitry for communications (e.g., directly or via network 106) with others of the audio device 150, the electronic device 104, the electronic device 110, the electronic device 115, and/or the server 120, the communications circuitry including, for example, one or more wireless interfaces, such as WLAN radios, cellular radios, Bluetooth radios, Zigbee radios, near field communication (NFC) radios, and/or other wireless radios. The audio device 150, the electronic device 104, the electronic device 110, the electronic device 112, and/or the electronic device 115 may each include a power sources such as a battery and/or a wired or wireless power source.

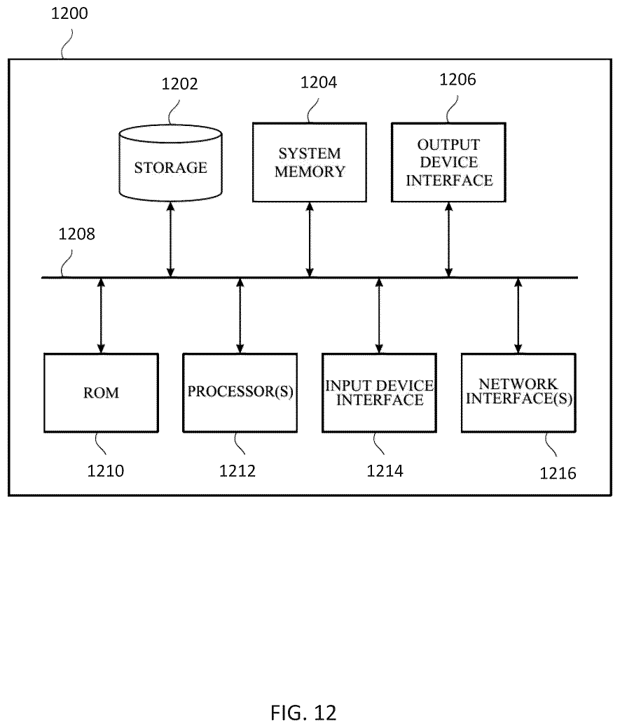

The electronic device 104 may be, for example, a smartphone, a portable computing device such as a laptop computer, a peripheral device (e.g., a digital camera, headphones, another audio device, or another media output device), a tablet device, a wearable device such as a smart watch, a smart band, and the like, any other appropriate device that includes, for example, processing circuitry and/or communications circuitry for processing microphone signals including audio content received from the physical environment around the electronic device 104, for providing sound levels to one or more other devices and/or servers, and/or for generating predictive guidance based on sound measurements. In FIG. 1, by way of example, the electronic device 104 is depicted as a mobile smartphone device. In one or more implementations, the electronic device 104 and/or the audio device 150 may be, and/or may include all or part of, the electronic device discussed below with respect to the electronic system discussed below with respect to FIG. 12.

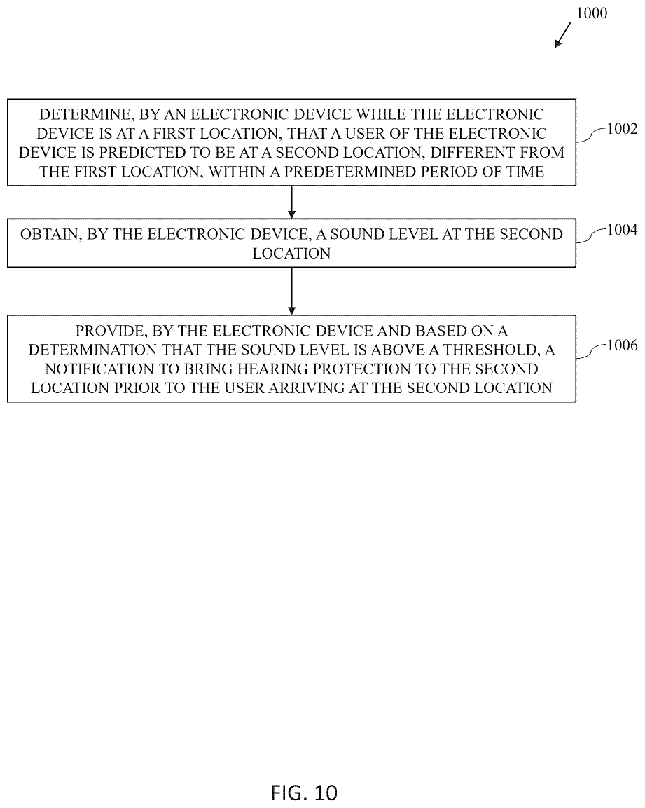

The electronic device 115 may be, for example, desktop computer, a portable computing device such as a laptop computer, a smartphone, a peripheral device (e.g., a digital camera, headphones, another audio device, or another media output device), a tablet device, a wearable device such as a watch, a band, and the like. In FIG. 1, by way of example, the electronic device 115 is depicted as a desktop computer. The electronic device 115 may be, and/or may include all or part of, the electronic system discussed below with respect to FIG. 10.

The electronic device 110 may be, for example, a smartphone, a portable computing device such as a laptop computer, a peripheral device (e.g., a digital camera, headphones, another audio device, or another media output device), a tablet device, a wearable device such as a smart watch, a smart band, and the like, any other appropriate device that includes, for example, processing circuitry and/or communications circuitry for processing microphone signals including audio content received from the physical environment around the electronic device 110, for providing sound levels to one or more other devices and/or servers, and/or for generating predictive guidance based on sound measurements. In FIG. 1, by way of example, the electronic device 110 is depicted as a mobile smartphone device. In one or more implementations, the electronic device 110 may be and/or may include all or part of, the electronic device discussed below with respect to the electronic system discussed below with respect to FIG. 10.

The electronic device 112 may be, for example, a smartphone, a portable computing device such as a laptop computer, a peripheral device (e.g., a digital camera, headphones, another audio device, or another media output device), a tablet device, a wearable device such as a smart watch, a smart band, and the like, any other appropriate device that includes, for example, processing circuitry and/or communications circuitry for processing microphone signals including audio content received from the physical environment around the electronic device 112, for providing sound levels to one or more other devices and/or servers, and/or for generating predictive guidance based on sound measurements. In FIG. 1, by way of example, the electronic device 112 is depicted as a smart watch that is worn on a wrist of the user 101. In one or more implementations, the electronic device 112 may be and/or may include all or part of, the electronic device discussed below with respect to the electronic system discussed below with respect to FIG. 12.

In one or more implementations, the electronic device 104, the audio device 150, and/or the electronic device 112 may be associated with the same user account (e.g., the electronic device 104, the audio device 150, and/or the electronic device 112 may be registered, such as at the servers 120, to a user account of the user 101). In one or more implementations, the electronic device 110 may be associated with a different user account (e.g., the electronic device 110 may be registered to a second user account of a second user, different from the user 101). The electronic device 110 may be unassociated with the user account of the user 101 and/or any other user accounts associated with the user 101, in one or more implementations.

The server 120 may form all or part of a network of computers or a group of servers 130, such as in a cloud computing or data center implementation. For example, the server 120 stores data and software, and includes specific hardware (e.g., processors, graphics processors and other specialized or custom processors) processing graphics, images, video, audio and/or multi-media files. In an implementation, the server 120 may function as a cloud storage server.

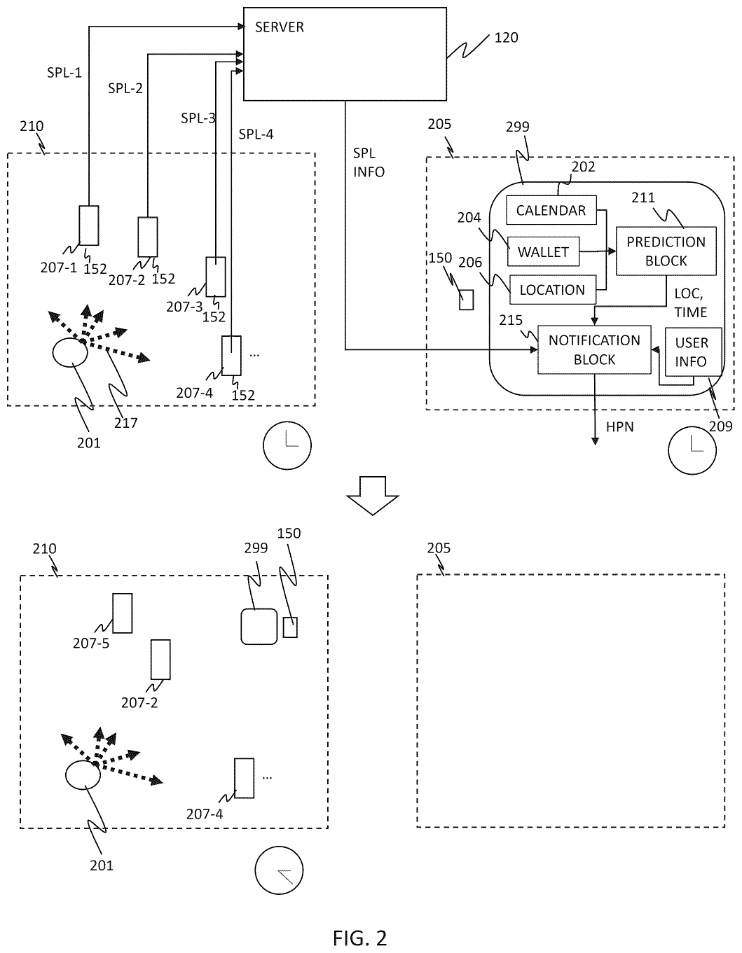

FIG. 2 illustrates an example use case in which an electronic device 299 (e.g., a smart watch, such as the electronic device 112, or a smartphone, such as the electronic device 104) is located a first location 205 (e.g., a location associated with the user 101, such as the home, office, or gym of the user, or any other location). In the example of FIG. 2, the electronic device 299 obtains a predicted future location (LOC) of the electronic device 299 and/or the user 101. For example, the electronic device 299 may store user-specific information for a user (e.g., user 101) of the electronic device 299. For example, the user-specific information may include calendar information 202 (e.g., calendared events, reminders, meetings, flights, etc.), wallet information 204 (e.g., payment information, purchase information, ticketed event information, or the like), location information 206 (e.g., a current location of the electronic device 299 and/or a location history of the electronic device 299), and/or other user information 209 (e.g., user hearing threshold preferences, and/or user hearing conditions, such as tinnitus).

As shown in FIG. 2, the electronic device 299 may provide the calendar information 202, the wallet information 204, and/or the location information 206 to a prediction block 211. For example, the prediction block may include one or more machine learning modules that have been trained to predict a future location, such as location 210, of the electronic device 299 and/or the user 101 at one or more future times. For example, the prediction block 211 may predict, based on a calendared dinner at a particular restaurant in the calendar information 202, that the electronic device 299 and/or the user 101 are predicted to be at the particular restaurant during a calendared time period of the calendared dinner. As another example, the prediction block 211 may predict, based on a ticket for a ticketed event (e.g., a concert, a play, a performance, a sporting event, a car race, etc.) in the wallet information 204, that the electronic device 299 and/or the user 101 are predicted to be at the ticketed event during a scheduled time of the ticketed event. As another example, the prediction block 211 may predict, based on a location history of the electronic device 299, that the electronic device 299 and/or the user 101 are predicted to be at a particular location (e.g., a workplace such as an office, a store, a construction site, a gym, a studio, a school, a classroom, a cafe, a restaurant, etc.) during a particular time period on a particular day of the week that the electronic device 299 and/or the user 101 have regularly or repeatedly visited that particular location in the past.

As shown, the prediction block 211 may predict a future location (LIC) and time information (TIME) associated with the predicted location. For example, the time information may include a predicted period of time during which the electronic device 299 and/or the user 101 are predicted to be at the predicted location, a predicted time of arrival at the predicted location, a predicted time a departure from a current location for the predicted location, and/or a predicted time of departure from the predicted location. In the example of FIG. 2, the predicted location and/or the predicted time may be provided to a notification block 215. As shown, the notification block 215 may obtain (e.g., responsive to receiving the predicted location and/or predicted time) sound information (e.g., sound pressure level (SPL) information, such as a sound level or a sound pressure level) for the predicted location. In the example of FIG. 2, the prediction block 211 and the notification block 215 are depicted as separate processing blocks, each of which may be implemented in hardware or software. However, in other implementations, the prediction block 211 and the notification block 215 may be part of a common hardware and/or software processing block, and may the operations of the prediction block 211 and the notification block 215 may be performed in series or partly or completely in parallel.

In the example of FIG. 2 the sound information (e.g., SPL INFO) obtained by the notification block 215 may be real-time sound information for the location 210. For example, one or more other electronic devices, such as electronic device 207-1, 207-2, 207-3, and 207-4 each having microphones 152, may be at the location 210 at or near the time (e.g., 3 pm in the example of FIG. 2) at which the notification block receives the predicted location. As shown, the one or more other electronic devices may each provide a sound level measurement (e.g., SPL-1, SPL-2, SPL-3, and SPL-4 from electronic devices 207-1, 207-2, 207-3, and 207-4 respectively), such as to a server, such as server 120. One or more of the electronic devices 207-1, 207-2, 207-3, and 207-4 may be portable electronic devices of other users (e.g., smartphone, smart watches, headphones, earbuds, tablets, laptops, such as the electronic device 110 or FIG. 1). One or more others of the electronic devices 207-1, 207-2, 207-3, and 207-4 may be fixed devices at the location 210, such as security cameras, smart speakers, streaming webcams, smart televisions, or any other fixed devices having one or more microphones that can sample the sound and/or determine the sound level at the location 210.

As shown, one or more sound sources, such as sound source 201, at the location 210 may generate noise or other sound within or around the location 210. In various examples, location 210 may include an indoor environment that is substantially enclosed by walls, doors, and/or windows, or may be an outdoor environment. For example, indoor environments may include restaurants, bars, clubs, arenas, theaters, buildings, rooms, factories, vehicles, watercraft, aircraft, shopping malls, warehouses, airports, and/or any other indoor venue in which one or more sources of sound are located. As examples, outdoor noise environments may include stadiums, theaters, concert venues, sporting events, auto racing events, airports, highways, parks, parking lots, parking garages, streets, sidewalks, and/or any other outdoor, open-air, unenclosed, or partially unenclosed environment in which one or more sources of sound are located.

Although a single sound source 201 is depicted in FIG. 2, it is appreciated that the sound source 201 may represent one sound source, two source sound sources, more than two sound sources, tens of sound sources, hundreds of sound sources, thousands of sound sources, or any other number of sound sources that individually and/or cumulatively contribute to the sound level at the location 210 and/or at any particular sub-location within the location 210. As examples, the sound source 201 may be a person, a loudspeaker, a machine, a vehicle, an aircraft, a watercraft, a siren, a crowd of people, a megaphone, a bullhorn, an appliance, and/or any other sound-generating device, component, object, and/or being.

As shown in FIG. 2, the sound source 201 may generate sound 217 that is received at a microphone 152 of each of the electronic devices 207-1, 207-2, 207-3, and 207-4. Each of the electronic devices 207-1, 207-2, 207-3, and 207-4 may determine a sound level (e.g., a sound pressure level, or SPL) of the sound 217 at the location of that device, and may provide the sound level to a server, such as server 120. As shown, the server 120 may provide sound information (e.g., SPL info) to the electronic device 299 based on the sound levels received from the electronic devices 207-1, 207-2, 207-3, and 207-4 that are at the location 210. For example, the sound information may be provided from the server 120 responsive to a request for the sound information for the location 210 from the electronic device 299 (e.g., the request sent to the server responsive to the determination, by the prediction block 211, that the electronic device 299 and/or the user 101 is predicted to be at the location 210).

In one or more implementations, the notification block 215 may compare a sound level received in the sound information from the server 120 for the location 210 to a threshold (e.g., a threshold sound level). Responsive to determining that the sound level at the location 210 exceeds the threshold, the notification block 215 may generate a hearing protection notification (HPN). For example, the hearing protection notification may be a notification to bring hearing protection to the location 210.

For example, the audio device 150 (e.g., headphones or earbuds having noise cancelling capabilities and having previously paired with the electronic device 299) may be located at the location 205 at the time when the notification block 215 generates the hearing protection notification. The hearing protection notification may include a notification or reminder to bring the audio device 150 from the location 205 to the location 210 (e.g., and to activation noise cancellation operations of the audio device 150).

For example, as shown in FIG. 2, at a later time (e.g., 3:20 pm), the electronic device 299 and the audio device 150 (e.g., and the user 101 thereof) may be at the location 210. In this example, when or before the user 101 arrives at or enters the location 210, the user 101 can don the audio device 150 and activate noise cancellation operations to reduce the amount of the sound 217 from the sound source 201 that reaches the cars of the user 101. As shown, some or all of the electronic devices (e.g., the electronic device 207-2 and the electronic device 207-4) that provided the sound level information upon which the hearing protection notification was based may still be at the location 210 when the electronic device 299 (e.g., and the audio device 150 and the user 101) arrive at the location 210. As shown, one or more additional electronic devices (e.g., the electronic device 207-5) may also be at the location 210 when the electronic device 299 (e.g., and the audio device 150 and the user 101) arrive at the location 210. In various examples, the hearing protection notification may be output by the electronic device 299 using a display of the electronic device 299, one or more speakers of the electronic device 299, one or more haptic components of the electronic device 299, and/or any other output component of the electronic device 299.

As shown in FIG. 2, the hearing protection notification may be provided by the electronic device 299 while the electronic device 299 (e.g., and the user 101 and the audio device 150) is at the location 205, separate from the location 210. In one or more other use cases, the hearing protection notification may be provided by the electronic device 299 at any of various other times prior to the arrival of the electronic device 299 (e.g., and the user 101 and the audio device 150) at the location 210.

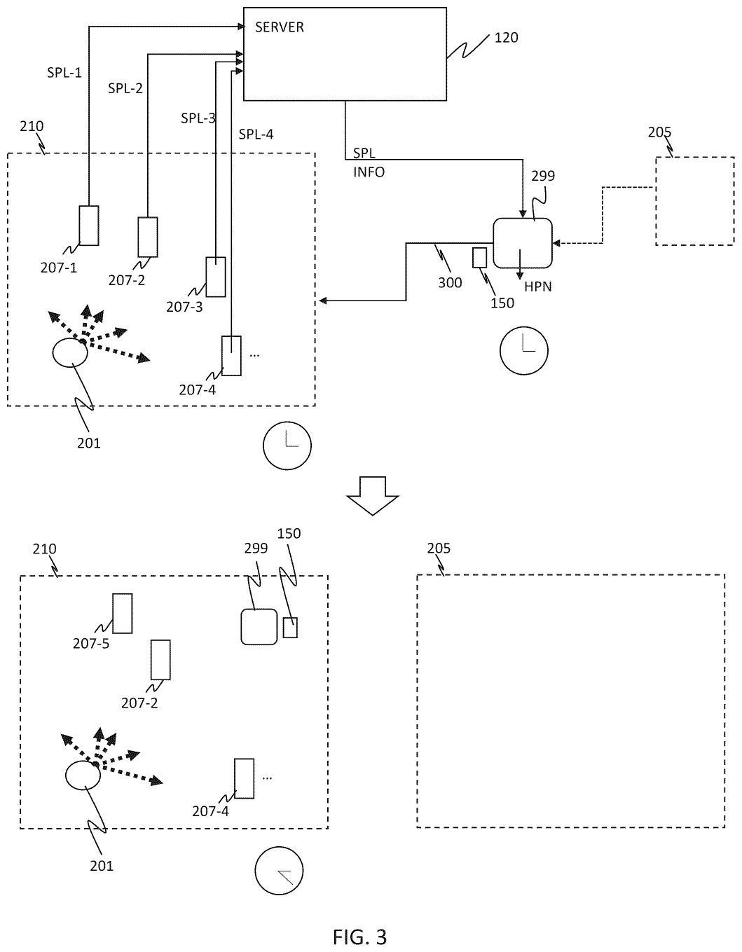

For example, FIG. 3 illustrates an example use case in which the electronic device 299 provides a hearing protection notification while the electronic device 299 (e.g., and the user 101 and the audio device 150) is on the way to the location 210 (e.g., en route along a route 300, such as between the location 205 and the location 210). For example, the use case of FIG. 3 may occur while the user 101 is driving, walking, or otherwise moving toward the location 210 (e.g., while carrying or wearing the electronic device 299 and/or the audio device 150). In this example use case, the prediction block 211 (see, e.g., FIG. 2) may predict that the user is predicted to be at the location 210 in the near future (e.g., within minutes or hours) based (e.g., in part) on location information 206 (e.g., global positioning system (GPS) information, and/or inertial measurement unit (IMU) information) that indicates that the electronic device 299 is en route to the location 210. For example, the prediction block 211 may recognize that the current location and motion of the electronic device 299 matches a portion of a route 300 previously, frequently, or regularly travelled by the user 101 (e.g., while carrying or wearing the electronic device 299 and/or the audio device 150) to the location 210. In this example, the hearing protection notification may include a reminder to not forget the audio device 150 in the car when entering the location 210. In this example, when or before the user 101 arrives at or enters the location 210, the user 101 can don the audio device 150 and activate noise cancellation operations to reduce the amount of the sound 217 from the sound source 201 that reaches the cars of the user 101.

In the examples of FIGS. 2 and 3, the sound level at the location 210 is measured concurrently with (e.g., in real time with) the generation of the hearing protection notification by the electronic device 299. However, in one or more other use cases, the sound level at a location, such as the location 210, may be an expected sound level at the time during which the electronic device 299 is predicted to be at the location 210, based on one or more previous measurements of the sound level at the location 210.

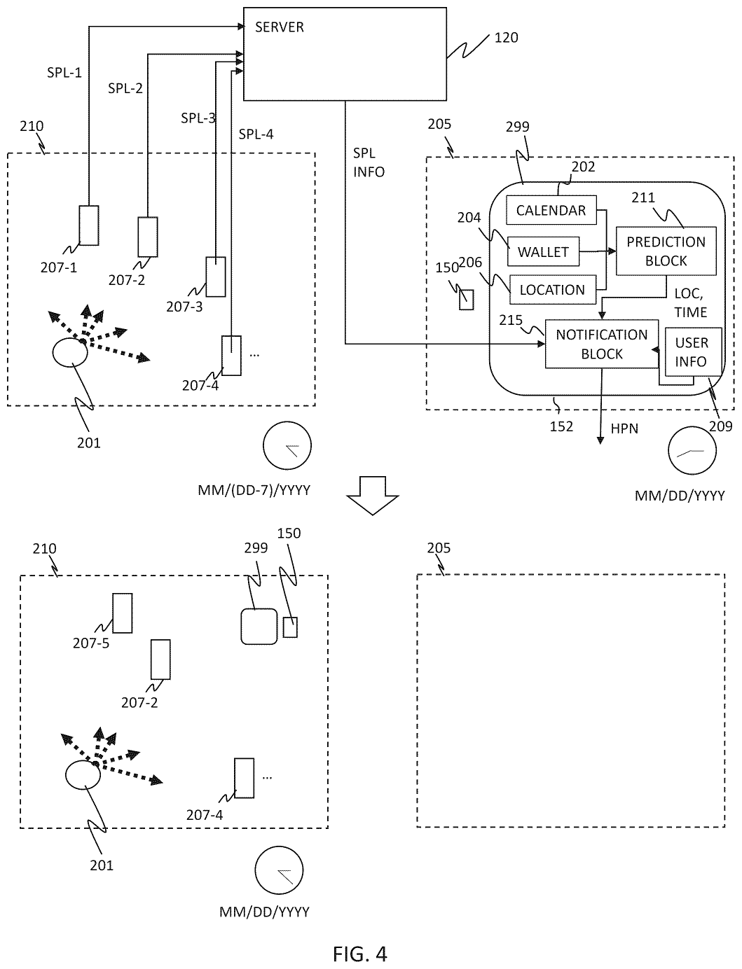

For example, FIG. 4 illustrates a use case in which the electronic devices (e.g., electronic devices 207-1, 207-2, 207-3, and 207-4) that provide the sound level measurements at the location 210 were previously at the location 210 at a previous time associated with a future time (e.g., the predicted time generated by the prediction block 211) at which the electronic device 299 (e.g., and the user 101) is predicted to be at the location 210. In the example of FIG. 4, the electronic devices 207-1, 207-2, 207-3, and 207-4 measure the sound level at the location 210 at a time (e.g., 3:20 pm) that is a week before (e.g., on a date of MM/DD-7/YYYY) the predicted arrival (e.g., at 3:20 pm on MM/DD/YYYY) of the electronic device 299 and/or the user 101 at the location 210.

In this example, the measurement of the sound level occurs once on the same day of the week at the same time of day as the expected arrival of the electronic device 299 and/or the user 101. However, it is appreciated that the expected sound level at the location 210 at the same time of day as the expected arrival of the electronic device 299 and/or the user 101 at the location 210 may be aggregated over several prior measurements at that time on that day of the week. It is also appreciated that the example of the measurement on the same day of the week is also illustrative, and that the measurements may have been obtained and/or aggregated from the same day of the month, the same day of the year, the day of a same event, or any other prior time period that is associated with the predicted time of arrival of the electronic device 299 and/or the user 101 at the location 210.

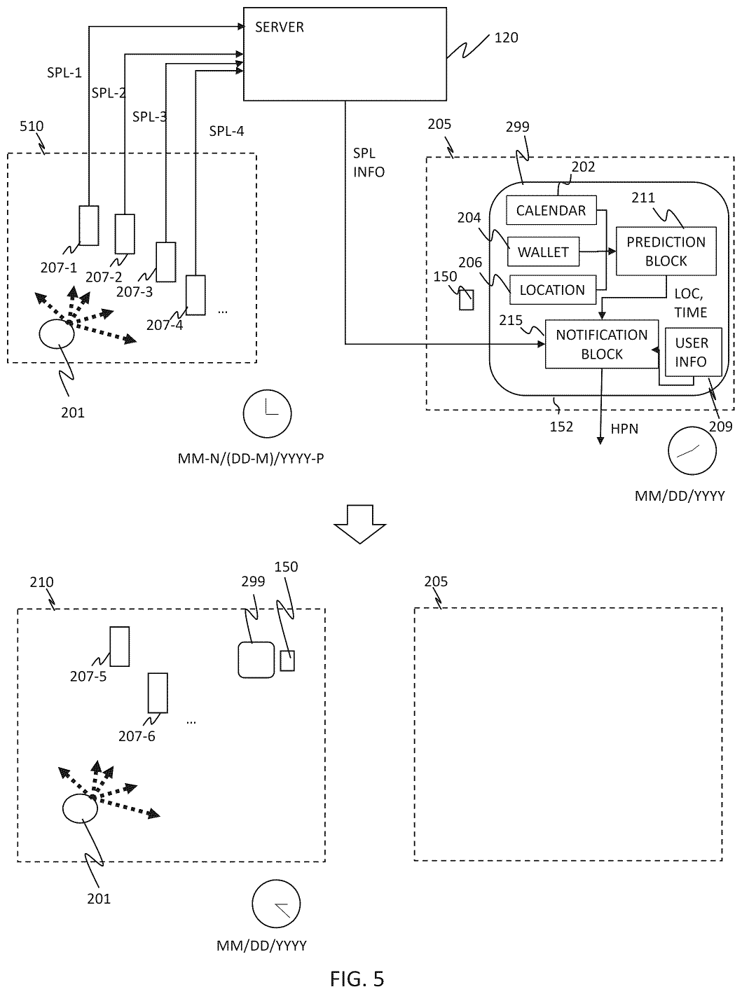

In the examples of FIGS. 2-4, the sound level at the location 210 (at the predicted time during which the electronic device 299 and/or the user 101 is predicted to be at the location 210) is measured at the location 210. However, in one or more other use cases, the sound level at a location, such as the location 210, may be an expected sound level at the time during which the electronic device 299 is predicted to be at the location 210, based on one or more previous measurements of the sound level at another location. For example, FIG. 5 illustrates an example in which the sound level at the location 210 is an expected sound level that is based on a sound level measured by one or more other devices (e.g., the electronic device 207-1, 207-2, 207-3, and 207-4) at a location 510, different from the location 210. For example, the environmental context at the location 510 at a previous time may be expected to be replicated at the location 210 at a future time. As examples, replicating the environmental context may include the same sound source moving from the location 510 to the location 210, or may include another sound source, of the same type as the sound source at the location 510, being at the location 210.

For example, as shown in FIG. 5, the same sound source 201 that is expected to be at the location 210 at the time when the electronic device 299, the user 101, and/or the audio device 150 are predicted to be at the location 210 may have been at the location 510 at an earlier time (e.g., 3 pm on a date MM-n/DD-mM/YYYY-p, where n is a number of months, m is a number of days, and p is a number of years) prior to the time of prediction (e.g., 2:40 pm on a date MM/DD/YYYY) by the prediction block 211 and prior to the time of arrival (e.g., 3:20 pm on the date MM/DD/YYYY) at the location 210. For example, the sound source 201 in this use case may be a touring band, concert group, show, race, team, sporting event, protest, demonstration, or other mobile sound-generating source that previously appeared or performed at the location 210 and that is scheduled to appear or perform at the location 210.

For example, the sound source 201 may be a rock band that played a concert at an arena corresponding to the location 510 in one city on a Friday night, and that is scheduled to play a concert at an arena corresponding to the location 210 on the following Saturday night or any other day or night following the Friday night performance at the location 510. In this example, the sound level expected at the location 210 may be expected to be the same as, or similar to, the sound level that occurred at the location 510. As shown, a different set of electronic devices (e.g., electronic device 207-5 and electronic device 207-6) from the electronic devices that measured the sound level at the location 510 may be at the location 210 while the electronic device 299, the user 101, and/or the audio device 150 are at the location 210. In this example, the electronic device 299 provides a hearing protection notification, based on the sound level measured at the location 210, prior to arrival of the electronic device 299, the audio device 150, and/or the user 101 at the location 210. The audio device 150 may then be used as a hearing protection device while the user is at the location 210, by cancelling or otherwise preventing some of the sound from the sound source 201 from reaching the user's ears.

In the example of FIG. 5, the same sound source 201 that is expected to be at the location 210 at the time when the electronic device 299, the user 101, and/or the audio device 150 are predicted to be at the location 210 may have been at the location 510 at an earlier time. In one or more other examples, a sound source of the same type that is expected to be at the location 210 at the time when the electronic device 299, the user 101, and/or the audio device 150 are predicted to be at the location 210 may have been at the location 510 at an earlier time. For example, a band or other source of sound in a sound category or sound type (e.g., a rock band, a rhythm and blues artist, a heavy metal band, an orchestra, a rap group, a classical music ensemble, a football game, a baseball game, a hockey game, a golf game, etc.) may have been at the location 510 while the devices 207-1, 207-2, 207-3, and 207-4 were at the location 510 making sound level measurements. The electronic device 299 may provide a hearing protection notification or other predictive guidance based on a determination that another source of sound (e.g., a different band in the same category, or a different football team) in the same sound category or having the same sound type is expected to be at the location 210 at the time when the electronic device 299, the user 101, and/or the audio device 150 are predicted to be at the location 210.

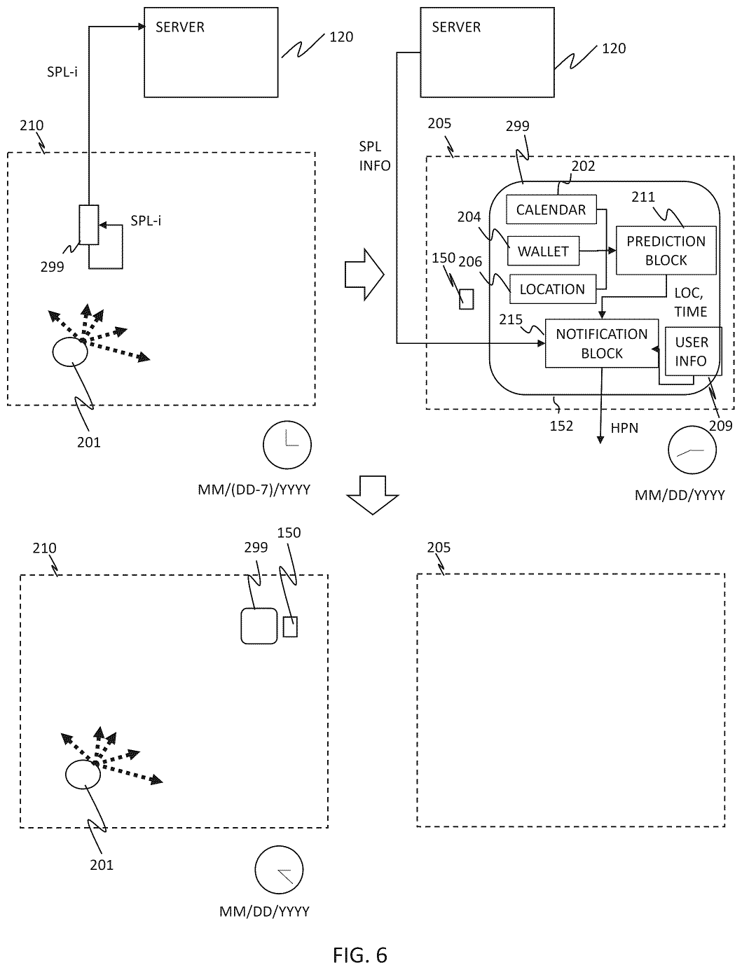

In the examples of FIGS. 2-5, the sound level at the location 210 at the predicted time during which the electronic device 299 and/or the user 101 is predicted to be at the location 210 is measured at the location 210 by one or more other electronic devices (e.g., one or more other electronic devices associated with other users and/or other user accounts). However, in one or more other use cases, the sound level at a location, such as the location 210, may be an expected sound level at the time during which the electronic device 299 is predicted to be at the location 210, based on one or more previous measurements of the sound level at that location by the electronic device 299 itself and/or by one or more other electronic devices of the user 101 of the electronic device 299. For example, FIG. 6 illustrates an example in which the sound level at the location 210 is an expected sound level that is based on a sound level measured by the electronic device 299 itself.

As shown, the electronic device 299 may have previously been at the location 210 at a previous time associated with a future time (e.g., the predicted time generated by the prediction block 211) at which the electronic device 299 (e.g., and the user 101) is predicted to be at the location 210. In the example of FIG. 6, the electronic devices 299 measures the sound level at the location 210 at a time (e.g., 3:20 pm on a date of MM/DD-7/YYYY) that is a week before a predicted future arrival (e.g., at 3:20 pm on MM/DD/YYYY) of the electronic device 299 and/or the user 101 at the location 210. For example, the user 101 may carry the electronic device 299 to the same worksite, the same cafe, the same restaurant, the same class, the same gym, or any other location at the same time each week, on the same day of each month, or on the same day of each year (as examples).

In this example, the measurement of the sound level by the electronic device 299 occurs once on the same day of the week at the same time of day as the expected future arrival of the electronic device 299 and/or the user 101. However, it is appreciated that the expected sound level at the location 210 at the same time of day as the expected arrival of the electronic device 299 and/or the user 101 at the location 210 may be aggregated over several prior measurements by the electronic device 299 and/or one or more other electronic devices associated with (e.g., registered to) a user account of the user 101 at that time on that day of the week. It is also appreciated that the example of the measurement by the electronic device 299 on the same day of the week is also illustrative, and that the measurements may obtained and/or aggregated from the same day of the month, the same day of the year, the day of a same event any other prior time period that is associated with the predicted future time of arrival of the electronic device 299 and/or the user 101 at the location 210.

As shown in FIG. 6, the sound level (e.g., SPL-i) that is measured by the electronic device 299 at the location 210 may be provided to the server 120 and/or may be provided for storage locally at the electronic device 299 (e.g., for later use by the notification block 215). As shown, at the later time (e.g., 3:20 pm on the date MM/DD/YYYY), the user 101 may carry (e.g., responsive to the hearing protection notification at 2:45 pm on the date MM/DD/YYYY) the electronic device 299 and the audio device 150 (e.g., for hearing protection, such as in a noise cancellation mode) into the location 210. The audio device 150 may then be used as a hearing protection device while the user is at the location 210, by cancelling or otherwise preventing some of the sound from the sound source 201 from reaching the user's ears.

In the examples of FIGS. 2-6, the time at which the prediction block 211 predicts the future location of the electronic device 299 and/or the user 101, and the time at which the notification block 215 generate the hearing protection notification may be prior to, and near to (e.g., within minutes or hours) of the arrival of the electronic device 299 and/or the user 101 at the predicted location. In this way, an effective reminder to bring hearing protection can be provided to the user 101 at the time when the reminder is relevant to the user and the user still has time and opportunity to act on the reminder (e.g., by bringing the audio device 150 when the user 101 leaves their house or office, or enters the location 210).

In the examples of FIGS. 2-6, the predictive guidance that is provided by the electronic device 299 based on sound measurements is provided in the form of a hearing protection notification (e.g., a notification or reminder to bring hearing protection to a future location). In one or more other implementations, the predictive guidance may include a recommended location and/or a recommended route that is based on sound level measurements at the location and/or along the recommended route. For example, based on the sound level measurements by multiple electronic devices at multiple locations in a physical environment, a sound-level map of the physical environment may be generated (e.g., by the server 120 and/or by one or more of the devices making the measurements). Based on the sound-level map, an electronic device, such as the electronic device 299, may provide a recommendation of a location (e.g., a restaurant, a venue, a park, a cafe, or the like) or a sub-location within a location (e.g., a seat, section, or row within an arena or stadium, a section of a restaurant, a particular table at a restaurant such as an indoor table or an outdoor table, an area within an airport) with a measured or expected sound level that is desired by a user, such as the user 101. Based on the sound-level map, an electronic device, such as the electronic device 299, may also, or alternatively, provide a recommended route with a measured or expected sound level that is desired by a user, such as the user 101.

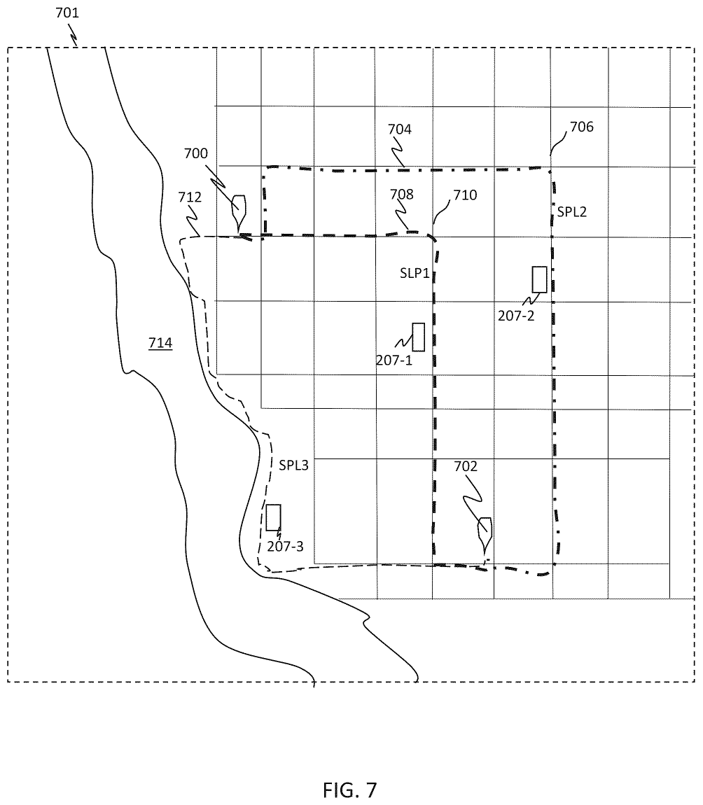

For example, FIG. 7 illustrates an example of recommended routes that may be provided (e.g., by the electronic device 299) between a location 700 and a location 702. As shown, one or more other electronic devices may be located at various respective locations in a physical environment represented by map 701. In one or more implementations, the map 701 may include a map of a physical environment including streets and geographical features such as a river 714, another body of water (e.g., a pond, a lake, a stream, an ocean), hills, parks, or the like. The map 701 may also include a third dimension corresponding to sound levels at various locations in the physical environment. For example, the sound levels may be represented as a heat map overlaid on the map of the physical environment.

The sound levels may be measured in real time and/or may have been measured and/or aggregated from measurements by multiple other electronic devices over time (e.g., hours, days, weeks, or years). The sound-level map may be a real-time, time varying, sound-level map of sound levels currently being measured (e.g., by the electronic device 207-1 on the street 710, by the electronic device 207-2 on the street 706, and/or by the electronic device 207-3 along the river 714), or the sound-level map may be generated for a particular time (e.g., the current time or a future time) based on prior measurements of the sound level at a time similar to the time for which the sound-level map is displayed (e.g., at the same time of day on the same day of the week, month or year). The sound-level map may include displayed sound level information on the map (e.g., in a heat-map style overlay on the map of the physical environment), or the sound-level-information may be stored as map metadata that is not visibly displayed on the map. In the example of FIG. 7, the map 701 is a map of an outdoor environment (e.g., a portion of a city or town). In other use cases, the map 701 may be a map of a portion of a venue, such as map of an interior of an arena, a theater, a stadium, museum, a restaurant, an airport, or the like).

In the example of FIG. 7, three routes 704, 708, and 712 between the location 700 and the location 702 are shown. As shown, the route 708 may be the shortest route between the location 700 and the location 702 and may have a sound level SPL1 (e.g., as measured in real time by the electronic device 207-1, and/or as expected based on previous measurements at a similar time by one or more other electronic devices along the street 710) along the route. The sound level SPL1 for the route 704 may be an average sound level along the route 704, a peak sound level along the route 704, or may be a set of sound levels at various locations along the route 704.

As shown, an additional route 704 between the location 700 and the location 702 may be provided that is longer in distance than the route 708, but that has a sound level SPL2 (e.g., as measured in real time by the electronic device 207-2, and/or as expected based on previous measurements at a similar time by one or more other electronic devices along the street 706) that is lower than the sound level SPL1 of the route 708. For example, the street 706 along which the route 704 progresses may have less traffic or less construction than the street 710 along which the route 708 progresses. As shown, an additional route 712 between the location 700 and the location 702 may be provided along the river 714, and that has a sound level SPL3 (e.g., as measured in real time by the electronic device 207-3, and/or as expected based on previous measurements at a similar time by one or more other electronic devices along the river 714) that is lower than the sound level SPL1 of the route 708 and lower than the sound level SPL2 of the route 704.

In one illustrative example, a user (e.g., user 101) of an electronic device (e.g., the electronic device 299) may request (e.g., via a user input to the electronic device 299) the quietest route (or a route with a sound level below a particular threshold) between the location 700 and the location 702, and the route 712 may be provided by the electronic device 299 (e.g., by the notification block 215 and based on the sound information received from the server 120). In another illustrative example, a user (e.g., user 101) of an electronic device (e.g., the electronic device 299) may request (e.g., via a user input to the electronic device 299) the quietest route (or a route with a sound level below a particular threshold) along paved streets between the location 700 and the location 702, and the route 704 may be provided by the electronic device 299 (e.g., by the notification block 215 and based on the sound information received from the server 120).

In one or more implementations, the provided route may be a route with a sound level that is below a threshold sound level. For example, the threshold sound level may be a sound level above which hearing damage is known to occur within a predetermined period of time. For example, the threshold noise level may be eighty decibels (dB), eight-five dB, ninety dB, ninety-five dB, one hundred dB, or another threshold noise level. In one or more implementations, the threshold noise level may be a threshold corresponding to a period of time. In one or more implementations, the period of time may depend on the decibel level of the threshold. As examples, the threshold noise level may be eighty dB for a period of several hours, eight-five dB for a period of one to two hours, ninety dB for a period of between fifteen minutes and one hour, ninety-five dB for a period of five minutes to fifteen minutes, one hundred dB for a period of one to five minutes, or another threshold noise level for another respective period of time.

In one or more implementations, the threshold sound level may be based, in part, on user-specific information, such as the user information 209 that is provided to the notification block 215. For example, the user-specific information may include a user-requested sound level in decibels, a user-requested sound range (e.g., quiet, moderate, loud) that the electronic device 299 internally maps to a range in decibels, or threshold that is specific to a hearing condition of the user (e.g., tinnitus, difficulty hearing, etc.).

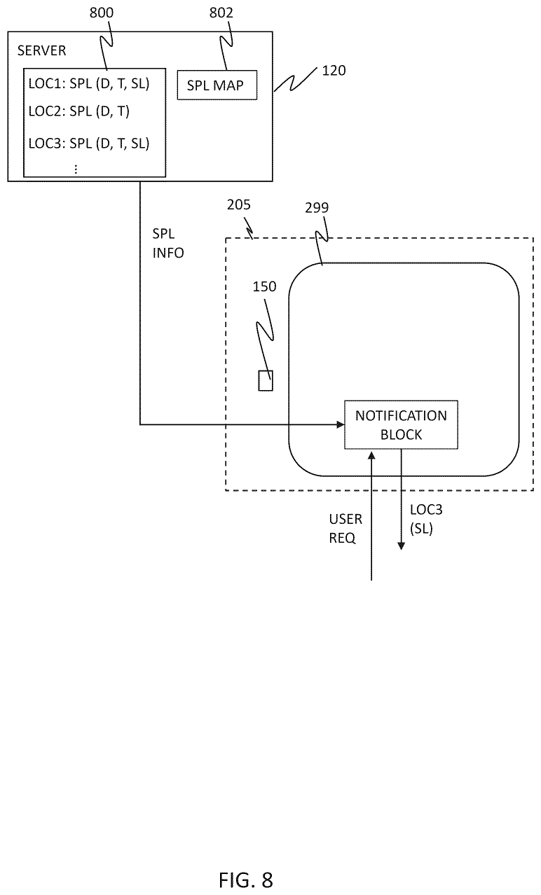

In the example of FIG. 7, a route between a location 700 and a location 702 is provided. However, in one or more other use cases, a recommended location (e.g., a recommendation of the location 702) may be provided by an electronic device, such as the electronic device 299. For example, FIG. 8 illustrates an example in which the server 120 stores and maintains a sound-level map (e.g., SPL map 802, such as an implementation of the map 701 of FIG. 7). FIG. 8 also illustrates how the server 120 may store sound-level information 800 (e.g., as part of the SPL map 802 or in addition to the SPL map 802) for one or more specific locations and/or sub-locations within a particular location). For example, the sound-level information 800 shown in FIG. 8 includes a sound level (SPL) for a first location (e.g., LOC1), a second location (e.g., LOC2), and a third location (e.g., LOC3).

As shown, the sound level (SPL) for the first location is stored as a function of date, D, time, T, and sub-location, SL, within the first location. As shown, the sound level (SPL) for the second location is stored as a function of date, D, and time, T (e.g., without any sub-location information). As shown, the sound level (SPL) for the third location may be stored as a function of date, D, time, T, and sub-location, SL, within the third location. Using the sound-level information and/or the SPL map 802, when a user requests (e.g., via a user input to the electronic device 299, such as via a touchscreen, or a voice input, such as a voice input to a virtual assistant or smart assistant at the electronic device 299) a quiet restaurant, a loud restaurant, a quiet park, a quiet cafe, a location within an airport in which a person with tinnitus can have a comfortable conversation, a loud restaurant that is suitable for kids, or a seat location within a stadium with a sound level below a threshold sound level (as illustrative examples), the electronic device 299 (e.g., the notification block 215) can provide (e.g., based on crowd sourcing of sound levels, which may be indexed by day and/or time, as described herein) a location recommendation that meets the user's request. In the example of FIG. 8, the electronic device 299 recommends the third location (e.g., LOC3) in response to a user request. In various implementations, a threshold sound level upon which a recommended location or route is based may be obtained, in part, on based on crowd-sourced information, a type of a point of interest, and/or metadata associated with media being played at the point of interest (e.g., a specific concert, band, act, performance, or the like). As examples, the threshold sound level for locating a relatively quiet location outside at an airport may be higher than the threshold sound level for locating a relatively quiet location inside an opera house, the threshold sound level for locating a relatively quiet location at a rock concert or a car race may be higher than the threshold for locating a relatively quiet location sound level in a restaurant, the threshold sound level for locating a relatively quiet location within a restaurant that is known (e.g., based on the crowd-sourced information) to be crowded and loud may be higher than the threshold sound level for locating a relatively quiet location in a fine-dining restaurant, or the threshold sound level for locating a relatively quiet outdoor walk may be lower than the threshold sound level for locating a relatively quiet indoor restaurant.

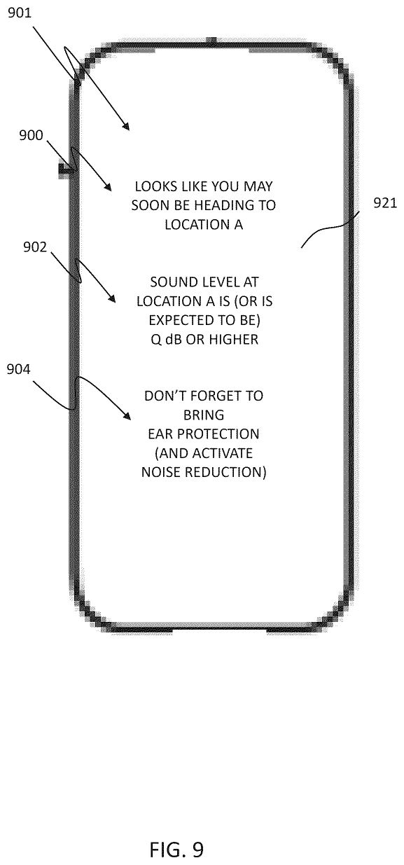

FIG. 9 illustrates an example of a hearing protection notification that may be provided by an electronic device, such as the electronic device 299, in accordance with one or more implementations. As shown in FIG. 9, a hearing protection notification 901 may be provided on a display 921 of the electronic device 299 in some implementations. As shown, the hearing protection notification may include a notification 900 of a predicted future location (e.g., location A in FIG. 9), a notification 902 of an expected sound level (e.g., Q dB in FIG. 9) at the predicted location, and a notification 904 (e.g., a reminder) to bring hearing protection to the location (e.g., and/or to activate noise cancellation operations of the hearing protection).

FIG. 10 illustrates a flow diagram of an example process 1000 for predictive guidance based on sound measurements, in accordance with implementations of the subject technology. For explanatory purposes, the process 1000 is primarily described herein with reference to the audio device 150 and electronic device 299 of FIGS. 2-9. However, the process 1000 is not limited to the audio device 150 and electronic device 299 of FIGS. 2-9, and one or more blocks (or operations) of the process 1000 may be performed by one or more other components of other suitable devices, including the electronic device 110, the electronic device 112, the electronic device 115, and/or the servers 120. Further for explanatory purposes, some of the blocks of the process 1000 are described herein as occurring in serial, or linearly. However, multiple blocks of the process 1000 may occur in parallel. In addition, the blocks of the process 1000 need not be performed in the order shown and/or one or more blocks of the process 1000 need not be performed and/or can be replaced by other operations.

As illustrated in FIG. 10, at block 1002, an electronic device (e.g., electronic device 299) may determine, while the electronic device is at a first location (e.g., location 205), that a user (e.g., user 101) of the electronic device is predicted to be at a second location (e.g., location 210), different from the first location, within a predetermined period of time (e.g., within minutes or hours of a current time). In one or more implementations, determining that the user is predicted to be at the second location may include determining that the user is predicted to be at the second location based on user information (e.g., calendar information 202, wallet information, 204, location information 206, and/or other user information 209) stored at the electronic device (e.g., and accessible only at the electronic device, for user privacy). For example, the user information may include at least one of: calendar information, purchase information, or location-history information for the user.

At block 1004, the electronic device may obtain a sound level (e.g., a sound pressure level, or SPL) at the second location. In one or more implementations, obtaining the sound level at the second location may include obtaining a measured current sound level based on a plurality of measurements of the sound level by a plurality of other respective electronic devices (e.g., electronic device 207-1, electronic device 207-2, electronic device 207-3, electronic device 207-4) that are at the second location at the time of the determining that that the user is predicted to be at the second location (e.g., as described herein in connection with FIGS. 2 and/or 3).

In one or more implementations, obtaining the sound level at the second location may include obtaining an expected sound level based on a plurality of measurements of the sound level by a plurality of other respective electronic devices (e.g., electronic device 207-1, electronic device 207-2, electronic device 207-3, electronic device 207-4) that were previously at the second location at a previous time associated with a future time (e.g., the same day of the week, the same day of the month, the same day of the year) at which the user is predicted to be at the second location (e.g., as described herein in connection with FIG. 4). For example, the future time may include a time of day or a day of the week (or a day of the month or a day of the year), and the expected sound level may include a historical average of the plurality of measurements (by the plurality of other respective electronic devices) of the sound level for the time of day or the day of the week.

In one or more implementations, obtaining the sound level at the second location may include obtaining an expected sound level based on a plurality of measurements of the sound level by a plurality of other respective electronic devices (e.g., electronic device 207-1, electronic device 207-2, electronic device 207-3, electronic device 207-4) that were previously at a third location (e.g., location 510) having an environmental context (e.g., the same sound source 201, such as the same band, play, tour, act, etc.) that is predicted to be present at the second location when the user is predicted to be at the second location (e.g., as described herein in connection with FIG. 5). In one or more implementations, obtaining the sound level at the second location may include obtaining an expected sound level based on one or more measurements of the sound level at the second location by the electronic device or another electronic device associated with the user of the electronic device at one or more previous times at which the electronic device or the other electronic device were at the second location (e.g., as described herein in connection with FIG. 6).

At block 1006, the electronic device may, based on a determination that the sound level is above a threshold, provide a notification (e.g., notification 901) to bring hearing protection to the second location prior to the user arriving at the second location. For example, the notification to bring hearing protection to the second location may include a notification to bring, to the second location, a hearing protection device (e.g., audio device 150) that has previously paired with the electronic device. For example, the hearing protection device may include a set of earbuds or headphones having a noise cancellation mode.

In one or more implementations, providing the notification may include providing the notification while the electronic device is at the first location and prior to a departure time of a departure of the user for the second location (e.g., to remind the user to bring the hearing protection before leaving their home, office, or other location at which the hearing protection may be located).

In one or more implementations, determining that the user is predicted to be at the second location may include determining that the electronic device is moving toward the second location from the first location, and providing the notification may include providing the notification while the electronic device is moving toward the second location and prior to the electronic device arriving at the second location (e.g., as described herein in connection with FIG. 3).

In one or more implementations, the notification may include a notification that the hearing protection is required at the second location. For example, the second location may be or include a workplace with a (e.g., crowd-sourced) sound level that is above a threshold that is considered safe (e.g., by an employer and/or a regulatory agency for the workplace) for long term unprotected exposure, and the notification may include a notification that the hearing protection is required for compliance with requirements and/or regulations at the workplace. In one or more implementations, the workplace (or another second location) may be geofenced, and a hearing protection notification may be issued and/or a hearing protection mode of earbuds or headphones of a user may be automatically activated when a user wearing the earbuds or headphones crosses into the geofenced workplace. The geofence may be a static geofence for a location that has a predictable known sound level (e.g., a factory or other manufacturing facility with loud machinery), or the geofence may be raised, lowered, and/or moved based on currently measured (e.g., crowd-sourced) sound levels.

In one or more implementations, an additional notification may be provided by the electronic device when the user leaves an environment in which the sound level is above the threshold. For example, the additional notification may include a notification that hearing protection can be removed or a hearing protection mode of earbuds or headphones can be deactivated or turned off. In the above example of a geofenced workplace (or other geofenced location), when a user that is wearing hearing protection leaves the workplace geofence, an additional notification may be provided with an invitation to remove or turn off the hearing protection. In one or more implementations, if the hearing protection is earbuds or headphones in a hearing protection and/or noise cancellation mode, the hearing protection may automatically turn of the hearing protection and/or noise cancellation mode when the user that is wearing the hearing protection leaves the workplace geofence.

In one or more implementations, when hearing protection is provided by a hearing protection mode of earbuds or headphones while a user is in a geofenced workplace, the user or an employer (with the explicit permission of the user) may track the sound exposure (e.g., the cumulative sound exposure) of the user over time while the user is within the geofenced workplace. The user or the employer (with the explicit permission of the user) may track the hearing protection that is provided by the earbuds to track a protection-corrected sound exposure (e.g., a user's particular exposure after mitigation using the hearing protection) over time. In this way, the user and/or the employer (with the explicit permission of the user) can be provided with a tool to prevent hearing damage over time in a noisy environment. In one or more implementations, the user may continue to track their own sound exposure (e.g., locally on their own device) outside of the workplace geofence.

In one or more implementations, the process 1000 may also include providing, by the electronic device and based on a subsequent determination that the sound level has fallen below the threshold, a notification that the hearing protection may be removed or deactivated. In one or more implementations, the second location may include a geofenced workplace, and the notification may include a notification that the hearing protection is required at the second location.

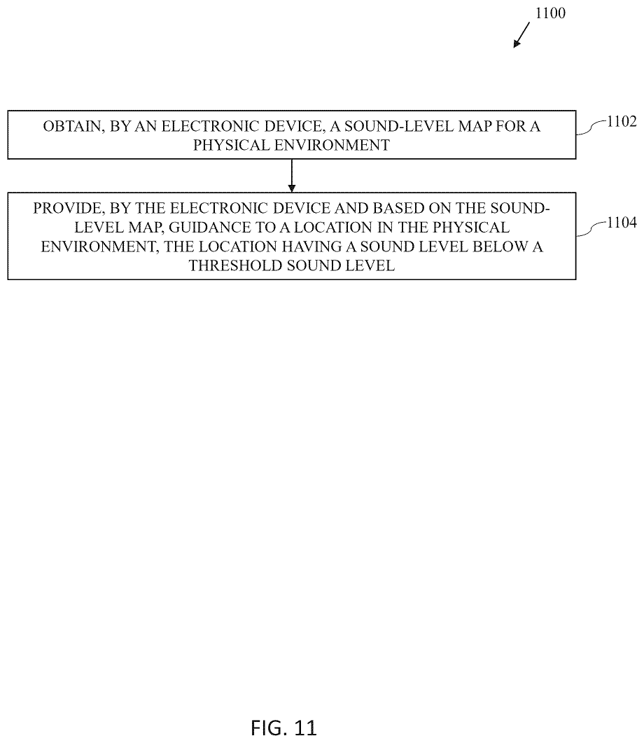

FIG. 11 illustrates a flow diagram of another example process 1100 for predictive guidance based on sound measurements, in accordance with implementations of the subject technology. For explanatory purposes, the process 1100 is primarily described herein with reference to the audio device 150 and electronic device 299 of FIGS. 2-9. However, the process 1100 is not limited to the audio device 150 and electronic device 299 of FIGS. 2-9, and one or more blocks (or operations) of the process 1100 may be performed by one or more other components of other suitable devices, including the electronic device 110, the electronic device 115, and/or the servers 120. Further for explanatory purposes, some of the blocks of the process 1100 are described herein as occurring in serial, or linearly. However, multiple blocks of the process 1100 may occur in parallel. In addition, the blocks of the process 1100 need not be performed in the order shown and/or one or more blocks of the process 1100 need not be performed and/or can be replaced by other operations.

As illustrated in FIG. 11, at block 1102, an electronic device (e.g., electronic device 299) may obtain a sound-level map (e.g., map 701 or SPL map 802) for a physical environment (e.g., an indoor environment or an outdoor environment). Obtaining the sound-level map may include obtaining some or all of the sound-level map from a server (e.g., server 120) that has received (e.g., crowd-sourced) sound level measurements and associated locations from multiple other electronic devices, and generated the sound-level map based on the received sound level measurements and associated locations.

At block 1104, the electronic device may provide, based on the sound-level map, guidance to a location (e.g., location 702 of FIG. 7 or LOC3 of FIG. 8) in the physical environment, the location having a sound level below a threshold sound level. For example, the threshold sound level may include a threshold sound level above which hearing damage is known to occur within a predetermined period of time. In one or more implementations, the threshold sound level is determined based on a user sound level request (e.g., in the user information 209). For example, the guidance to the location may be provided responsive to a user request for a quiet restaurant, a relatively quiet location within a concert venue, or the like as discussed herein.

In one or more implementations, the user sound level request may include a request for a venue, remote from a current location of the electronic device, having a sound level below the threshold sound level (e.g., as described herein in connection with FIG. 8). For example, the sound-level map may be a map of crowd-sourced sound levels at various physical locations including the venue, the request for the venue may include a request for a quiet restaurant (or other quiet venue), and the guidance may include a recommendation of the venue as the quiet restaurant. For example, the sound-level map may indicate that the venue is a quiet restaurant in some example use cases.

In one or more implementations, the user sound level request may include a request for a location (e.g., a sub-location) within a venue, within which the electronic device is located, having a sound level below the threshold sound level. In one or more implementations, the user sound level request may include an indication of a hearing condition of a user of the electronic device (e.g., the device may look up and use a threshold that is suitable for people with that hearing condition). In one or more implementations, the user sound level request may include a request for directions along a route (e.g., route 712) having a sound level below the threshold sound level (e.g., as described herein in connection with FIG. 7).

As described above, one aspect of the present technology is the gathering and use of data available from specific and legitimate sources for predictive guidance based on sound measurements. The present disclosure contemplates that in some instances, this gathered data may include personal information data that uniquely identifies or can be used to identify a specific person. Such personal information data can include audio data, voice samples, voice profiles, demographic data, location-based data, online identifiers, telephone numbers, email addresses, home addresses, biometric data or records relating to a user's health or level of fitness (e.g., vital signs measurements, medication information, exercise information), date of birth, or any other personal information.

The present disclosure recognizes that the use of such personal information data, in the present technology, can be used to the benefit of users. For example, the personal information data can be used for providing predictive guidance based on sound measurements.

The present disclosure contemplates that those entities responsible for the collection, analysis, disclosure, transfer, storage, or other use of such personal information data will comply with well-established privacy policies and/or privacy practices. In particular, such entities would be expected to implement and consistently apply privacy practices that are generally recognized as meeting or exceeding industry or governmental requirements for maintaining the privacy of users. Such information regarding the use of personal data should be prominently and easily accessible by users, and should be updated as the collection and/or use of data changes. Personal information from users should be collected for legitimate uses only. Further, such collection/sharing should occur only after receiving the consent of the users or other legitimate basis specified in applicable law. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations which may serve to impose a higher standard. For instance, in the US, collection of or access to certain health data may be governed by federal and/or state laws, such as the Health Insurance Portability and Accountability Act (HIPAA); whereas health data in other countries may be subject to other regulations and policies and should be handled accordingly.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the example of predictive guidance based on sound measurements, the present technology can be configured to allow users to select to “opt in” or “opt out” of participation in the collection and/or sharing of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing identifiers, controlling the amount or specificity of data stored (e.g., collecting location data at city level rather than at an address level or at a scale that is insufficient for facial recognition), controlling how data is stored (e.g., aggregating data across users), and/or other methods such as differential privacy.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data.

FIG. 12 illustrates an electronic system 1200 with which one or more implementations of the subject technology may be implemented. The electronic system 1200 can be, and/or can be a part of, the audio device 150, the electronic device 104, the electronic device 110, the electronic device 115, and/or the server 120 as shown in FIG. 1. The electronic system 1200 may include various types of computer readable media and interfaces for various other types of computer readable media. The electronic system 1200 includes a bus 1208, one or more processing unit(s) 1212, a system memory 1204 (and/or buffer), a ROM 1210, a permanent storage device 1202, an input device interface 1214, an output device interface 1206, and one or more network interfaces 1216, or subsets and variations thereof.

The bus 1208 collectively represents all system, peripheral, and chipset buses that communicatively connect the numerous internal devices of the electronic system 1200. In one or more implementations, the bus 1208 communicatively connects the one or more processing unit(s) 1212 with the ROM 1210, the system memory 1204, and the permanent storage device 1202. From these various memory units, the one or more processing unit(s) 1212 retrieves instructions to execute and data to process in order to execute the processes of the subject disclosure. The one or more processing unit(s) 1212 can be a single processor or a multi-core processor in different implementations.

The ROM 1210 stores static data and instructions that are needed by the one or more processing unit(s) 1212 and other modules of the electronic system 1200. The permanent storage device 1202, on the other hand, may be a read-and-write memory device. The permanent storage device 1202 may be a non-volatile memory unit that stores instructions and data even when the electronic system 1200 is off. In one or more implementations, a mass-storage device (such as a magnetic or optical disk and its corresponding disk drive) may be used as the permanent storage device 1202.

In one or more implementations, a removable storage device (such as a floppy disk, flash drive, and its corresponding disk drive) may be used as the permanent storage device 1202. Like the permanent storage device 1202, the system memory 1204 may be a read-and-write memory device. However, unlike the permanent storage device 1202, the system memory 1204 may be a volatile read-and-write memory, such as random access memory. The system memory 1204 may store any of the instructions and data that one or more processing unit(s) 1212 may need at runtime. In one or more implementations, the processes of the subject disclosure are stored in the system memory 1204, the permanent storage device 1202, and/or the ROM 1210 (which are each implemented as a non-transitory computer-readable medium). From these various memory units, the one or more processing unit(s) 1212 retrieves instructions to execute and data to process in order to execute the processes of one or more implementations.

The bus 1208 also connects to the input and output device interfaces 1214 and 1206. The input device interface 1214 enables a user to communicate information and select commands to the electronic system 1200. Input devices that may be used with the input device interface 1214 may include, for example, alphanumeric keyboards and pointing devices (also called “cursor control devices”). The output device interface 1206 may enable, for example, the display of images generated by electronic system 1200. Output devices that may be used with the output device interface 1206 may include, for example, printers and display devices, such as a liquid crystal display (LCD), a light emitting diode (LED) display, an organic light emitting diode (OLED) display, a flexible display, a flat panel display, a solid state display, a projector, or any other device for outputting information. One or more implementations may include devices that function as both input and output devices, such as a touchscreen. In these implementations, feedback provided to the user can be any form of sensory feedback, such as visual feedback, auditory feedback, or tactile feedback; and input from the user can be received in any form, including acoustic, speech, or tactile input.

Finally, as shown in FIG. 12, the bus 1208 also couples the electronic system 1200 to one or more networks and/or to one or more network nodes, such as the electronic device 110 shown in FIG. 1, through the one or more network interface(s) 1216. In this manner, the electronic system 1200 can be a part of a network of computers (such as a LAN, a wide area network (“WAN”), or an Intranet, or a network of networks, such as the Internet. Any or all components of the electronic system 1200 can be used in conjunction with the subject disclosure.

These functions described above can be implemented in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f), unless the element is expressly recited using the phrase “means for” or, in the case of a method claim, the element is recited using the phrase “step for”.

What is claimed is:

1. A method, comprising:
determining, by an electronic device while the electronic device is at a first location, that a user of the electronic device is predicted to be at a second location, different from the first location, within a predetermined period of time;
obtaining, by the electronic device, a sound level at the second location; and
providing, by the electronic device and based on a determination that the sound level is above a threshold, a notification to bring hearing protection to the second location prior to the user arriving at the second location.

2. The method of claim 1, wherein determining that the user is predicted to be at the second location comprises determining that the user is predicted to be at the second location based on user information stored at the electronic device.

3. The method of claim 2, wherein the user information comprises at least one of:
calendar information, purchase information, or location-history information for the user.

4. The method of claim 1, wherein obtaining the sound level at the second location comprises obtaining a measured current sound level based on a plurality of measurements of the sound level by a plurality of other respective electronic devices that are at the second location at the time of the determining that that the user is predicted to be at the second location.

5. The method of claim 1, wherein obtaining the sound level at the second location comprises obtaining an expected sound level based on a plurality of measurements of the sound level by a plurality of other respective electronic devices that were previously at the second location at a previous time associated with a future time at which the user is predicted to be at the second location.

6. The method of claim 5, wherein the future time comprises a time of day or a day of the week, and wherein the expected sound level comprises a historical average of the plurality of measurements of the sound level for the time of day or the day of the week.

7. The method of claim 1, wherein obtaining the sound level at the second location comprises obtaining an expected sound level based on a plurality of measurements of the sound level by a plurality of other respective electronic devices that were previously at a third location having an environmental context that is predicted to be present at the second location when the user is predicted to be at the second location.

8. The method of claim 1, wherein obtaining the sound level at the second location comprises obtaining an expected sound level based on one or more measurements of the sound level at the second location by the electronic device or another electronic device associated with the user of the electronic device at one or more previous times at which the electronic device or the other electronic device were at the second location.

9. The method of claim 1, wherein the notification to bring hearing protection to the second location comprises a notification to bring, to the second location, a hearing protection device that has previously paired with the electronic device.

10. The method of claim 9, wherein the hearing protection device comprises a set of earbuds having a noise cancellation mode.

11. The method of claim 1, wherein providing the notification comprises providing the notification while the electronic device is at the first location and prior to a departure time of a departure of the user for the second location.

12. The method of claim 1, wherein determining that the user is predicted to be at the second location comprises determining that the electronic device is moving toward the second location from the first location, and wherein providing the notification comprises providing the notification while the electronic device is moving toward the second location and prior to the electronic device arriving at the second location.

13. The method of claim 1, further comprising providing, by the electronic device and based on a subsequent determination that the sound level has fallen below the threshold, a notification that the hearing protection may be removed or deactivated.

14. The method of claim 1, wherein the second location comprises a geofenced workplace, and wherein the notification comprises a notification that the hearing protection is required at the second location.

15. A method, comprising:
obtaining, by an electronic device, a sound-level map for a physical environment; and
providing, by the electronic device and based on the sound-level map, guidance to a location in the physical environment, the location having a sound level below a threshold sound level.

16. The method of claim 15, wherein the threshold sound level comprises a threshold sound level above which hearing damage is known to occur within a predetermined period of time.

17. The method of claim 15, wherein the threshold sound level is determined based on a user sound level request.

18. The method of claim 17, wherein the user sound level request comprises a request for a venue, remote from a current location of the electronic device, having a sound level below the threshold sound level.

19. The method of claim 18, wherein the sound-level map comprises a map of crowd-sourced sound levels at various physical locations including the venue, wherein the request for the venue comprises a request for a quiet restaurant, and wherein the guidance comprises a recommendation of the venue as the quiet restaurant.

20. The method of claim 17, wherein the user sound level request comprises a request for a location within a venue, within which the electronic device is located, having a sound level below the threshold sound level.

21. The method of claim 17, wherein the user sound level request comprises an indication of a hearing condition of a user of the electronic device.

22. The method of claim 17, wherein the user sound level request comprises a request for directions along a route having a sound level below the threshold sound level.

23. The method of claim 15, wherein the sound-level map comprises a heat map of sound pressure levels at corresponding locations in the physical environment.

24. The method of claim 23, wherein the heat map is a time-dependent heat map in which the sound pressure levels vary in accordance with variations of sound in the physical environment.

25. An electronic device, comprising:
a memory; and
one or more processors configured to:
determine, while the electronic device is at a first location, that a user of the electronic device is predicted to be at a second location, different from the first location, within a predetermined period of time;
obtain a sound level at the second location; and
provide, based on a determination that the sound level is above a threshold and prior to the user arriving at the second location, a noise notification for the second location.

26. The electronic device of claim 25, wherein the one or more processors are configured to obtain the sound level responsive to the determination that the user is predicted to be at the second location, and wherein the noise notification comprises a notification to wear hearing protection at the second location.
